(19) United States
(12) Patent Application Publication
(10) Pub. No.: US 2010/0114053 A1
(43) Pub. Date: May 6, 2010

(54) BIRTHPAD

(76) Inventor: Deborah Kay Mandeville, Victoria (AU)

Correspondence Address: MILES & STOCKBRIDGE PC, 1751 PINNACLE DRIVE, SUITE 500, MCLEAN, VA, 22102-3833, US

(21) Appl. No.: 12/523,699

(22) PCT Filed: Jan. 16, 2008
(86) PCT No.: PCT/AU2008/000053

§ 371 Date: Jan. 11, 2010

(30) Foreign Application Priority Data
Jan. 17, 2007 (AU) 2007900319

(51) Int. Cl. A61F 13/45; A61F 7/02

(52) U.S. Cl. 604/385.06; 607/108

(57) ABSTRACT

The present invention relates broadly to a therapeutic cooling or heating pad or garment. One aspect of the invention relates to a therapeutic cooling or heating pad comprising a sanitary napkin and a cooling or heating agent coupled to or included within the sanitary napkin. Another aspect of the invention relates to a therapeutic cooling or heating garment comprising an undergarment and a cooling or heating agent coupled to or included within the undergarment.

### FIELD OF THE INVENTION

The present invention relates broadly to a therapeutic cooling or heating pad or garment.

### BACKGROUND TO THE INVENTION

After giving birth a woman may suffer from pain and bruising in the pelvic area. Women having had a caesarean section may also suffer from pain in the lower abdominal region. Pain medication may be used to relieve pain and treat bruising in these situations.

### SUMMARY OF THE INVENTION

According to one aspect of the invention, there is provided a therapeutic cooling or heating pad comprising:

a sanitary napkin; and

a cooling or heating agent coupled to or included within the sanitary napkin.

Preferably the cooling or heating agent is enclosed in one or more packs. More preferably the packs are disposable.

Preferably the sanitary napkin is disposable.

Preferably the sanitary napkin includes one or more pockets. More preferably the pockets are in the form of a sleeve formed in the sanitary napkin and having a closable opening at one end. Alternately the pockets are defined by a collapsible opening formed on an outward face of the sanitary napkin.

Preferably the one or more packs are carried within the one or more pockets. More preferably the one or more packs are removable from the one or more pockets. Even more preferably the one or more packs are replaceable.

Preferably the one or more pockets are located predominantly in the crotch region of the sanitary napkin.

Preferably the cooling or heating agent is reusable.

Generally the cooling agent includes a cooling gel, ice, a phase change material or salt solution. Generally the heating agent includes a phase change material or salt solution.

According to another aspect of the invention, there is provided a therapeutic cooling or heating garment comprising:

an undergarment; and

a cooling or heating agent coupled to or included within the undergarment.

Preferably the undergarment is in the form of underpants.

Preferably the cooling or heating agent is located near the pelvic or crotch area and/or lower abdominal region of the underpants.

Preferably the underpants are adapted to be closely fitted to the pelvic or crotch area and/or lower abdominal region of a human body.

Preferably the cooling or heating agent is enclosed in one or more packs.

Preferably the underpants include one or more pockets. More preferably the one of more pockets are located near the pelvic or crotch area and/or lower abdominal region of the underpants. Even more preferably the one or more packs are carried within the one or more pockets. Still more preferably the one or more packs are removable from the one or more pockets. Still even more preferably the one or more packs are replaceable.

Preferably the cooling or heating agent is reusable.

Generally the cooling agent includes a cooling gel, ice, a phase change material or salt solution. Generally the heating agent includes a phase change material or salt solution.

### BRIEF DESCRIPTION OF THE DRAWINGS

FIG. 1 Isometric view of one embodiment of the therapeutic cooling or heating pad before it is assembled.

FIG. 2 Cross-sectional view of the embodiment of the therapeutic cooling or heating pad shown in FIG. 1 after it is assembled.

FIG. 3 Isometric view of another embodiment of the therapeutic cooling or heating pad before it is assembled.

FIG. 4 Isometric view of yet another embodiment of the therapeutic cooling or heating pad before it is assembled.

FIG. 5 Isometric view of the embodiment of the therapeutic cooling or heating pad shown in FIG. 5 after it is assembled.

FIG. 6 Isometric view of an embodiment of the therapeutic cooling or heating garment before it is assembled.

FIG. 7 Isometric view of another embodiment of the therapeutic cooling or heating garment before it is assembled.

### DETAILED DESCRIPTION OF THE PREFERRED EMBODIMENTS

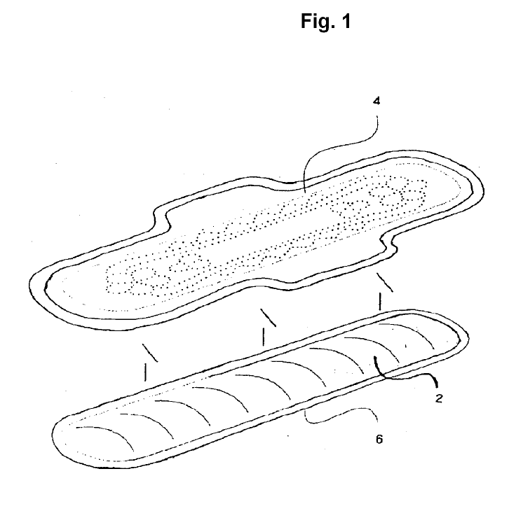

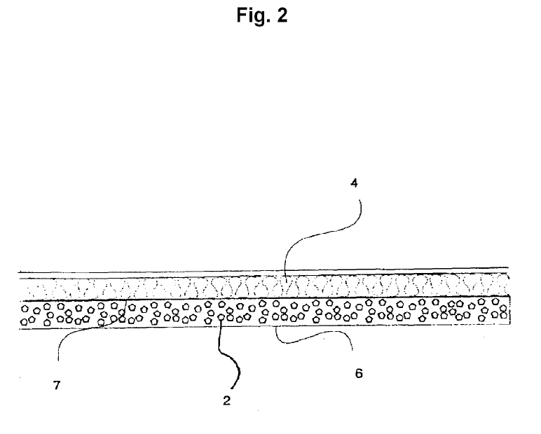

One aspect of the invention relates to a therapeutic cooling or heating pad. The therapeutic cooling or heating pad generally comprises a sanitary napkin and a cooling or heating agent included or integrated within the sanitary napkin. FIG. 1 shows how an embodiment of the therapeutic cooling or heating pad may be realised. In this embodiment, a pack 6 containing a cooling or heating agent 2 is attached to an outward face of a sanitary napkin 4. FIG. 1 shows the pack 6 and the sanitary napkin 4 in this embodiment as being separated before the therapeutic cooling or heating pad is assembled. FIG. 2 shows a partial cross-sectional view of the same embodiment of the therapeutic cooling or heating pad when assembled. The pack 6 may be attached to the sanitary napkin 4 using an adhesive strip, or other attaching means such as Velcro® (being a registered trade mark of Velcro Industries B.V.) tabs or dots. The sanitary napkin 4 may include a moisture barrier strip 7 to reduce the moisture absorbed by the sanitary napkin 4.

In this embodiment, and in others described below, the sanitary napkin 4 may be a sanitary towel, sanitary pad or menstrual pad. The sanitary napkin 4 may also be disposable. The cooling or heating agent 2 may be a phase change material or salt solution. A suitable cooling agent may also include ice or a cooling gel. The pack 6 containing the cooling or heating agent 2 may be disposable. The pack 6, including the cooling and heating agent 2, may also be removable from the sanitary napkin 4. Furthermore the pack 6, including the cooling and heating agent 2, may be reusable. For example, if ice is used as the cooling agent, the pack 6 may be cooled or frozen again after the pad's temperature rises above the desired temperature. Similarly, if a phase change material or salt solution is used as the heating agent, the pack 6 may be warmed or heated again after the pad's temperature drops below the desired temperature. The pack 6 containing the cooling or heating agent 2 may also be replaceable, if necessary, with another pack containing the same type or a different type of cooling or heating agent.

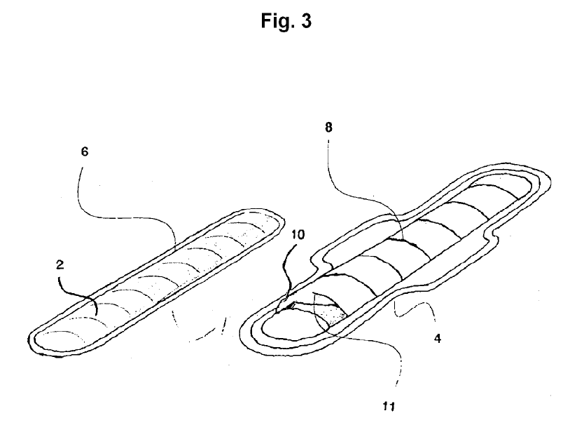

FIG. 3 shows how another embodiment of the therapeutic cooling or heating pad may be realised. In this embodiment, a pack 6 containing a cooling or heating agent 2 is put in a pocket or a sleeve 8 that is formed in the sanitary napkin 4 having a closable opening 10 at one end. The pocket or sleeve 8 may also be padded. The closable opening 10 may be closed and re-opened using an adhesive closure tab or Velcro® tabs or dots 11. FIG. 3 shows the pack 6 and the sanitary napkin 4 in this embodiment as being separated before the therapeutic cooling or heating pad is assembled.

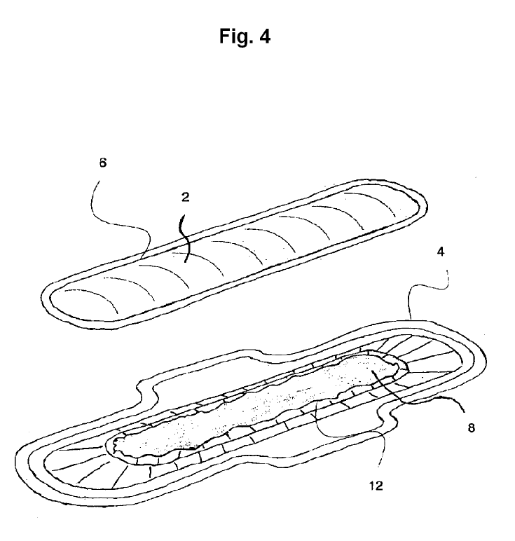

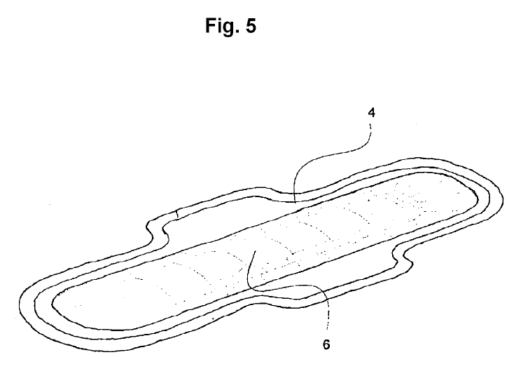

FIG. 4 shows how yet another embodiment of the therapeutic cooling or heating pad may be realised. In this embodiment, a pack 6 containing a cooling or heating agent 2 is put in a pocket or a sleeve 8 that is defined by a collapsible opening 12 formed on an outward face of the sanitary napkin 4. The collapsible opening 12 may be in a form of an elastic opening. FIG. 4 shows the pack 6 and the sanitary napkin 4 in this embodiment as being separated before the therapeutic cooling or heating pad is assembled. FIG. 5 shows an isometric view of this embodiment of the therapeutic cooling or heating pad when assembled.

In the embodiments where there is a pocket or sleeve for carrying the pack containing the cooling or heating agent, the location of the pocket may be predominantly in the crotch region of the sanitary napkin.

In some embodiments the sanitary napkin may have a reduced thickness compared to commonly used sanitary napkins. The reduced thickness makes heat transfer across the sanitary napkin more effective.

Another aspect of the invention relates to a therapeutic cooling or heating garment. The therapeutic cooling or heating garment generally comprises an undergarment and a cooling or heating agent included or integrated within the undergarment. The undergarment may be in the form of underpants or underwear. The undergarment may be designed to closely fit to one or more regions, such as the crotch, pelvic and/or lower abdominal area, of a human body.

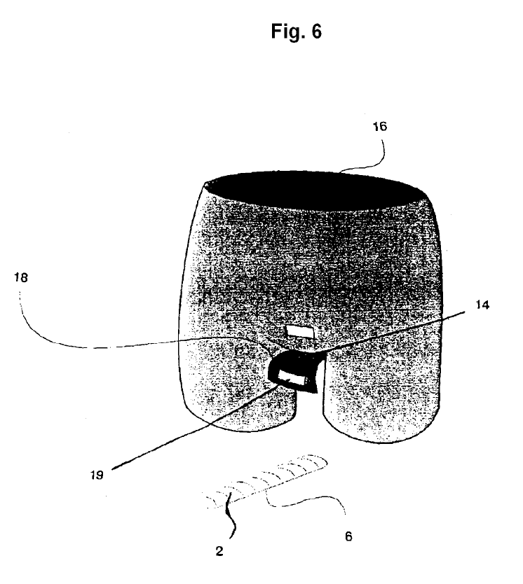

FIG. 6 shows an isometric view of how an embodiment of the therapeutic cooling or heating garment may be implemented. In this embodiment, a pack 6 containing a cooling or heating agent 2 is enclosed in a pocket or sleeve 14 of an undergarment such as Lycra® (being a registered trademark of Invista Technologies S.ar.l) underpants 16. The pocket or sleeve 14 may include a closeable opening 18. The closeable opening 18 may be closed and re-opened using Velcro® tabs or dots 19. FIG. 6 shows the pack 6 and the Lycra® underpants 16 in this embodiment as being separated before the therapeutic cooling or heating garment is assembled. As shown in FIG. 6 the location of the pocket or sleeve 14 in this embodiment is near the pelvic or crotch area of the Lycra® underpants 16.

The cooling or heating agent 2 of the therapeutic cooling or heating garment also shares similar features (such as the materials used and being reusable) to the cooling or heating agent of the therapeutic cooling or heating pad described above. The pack 6 containing the cooling or heating agent 2 may also be disposable and/or replaceable.

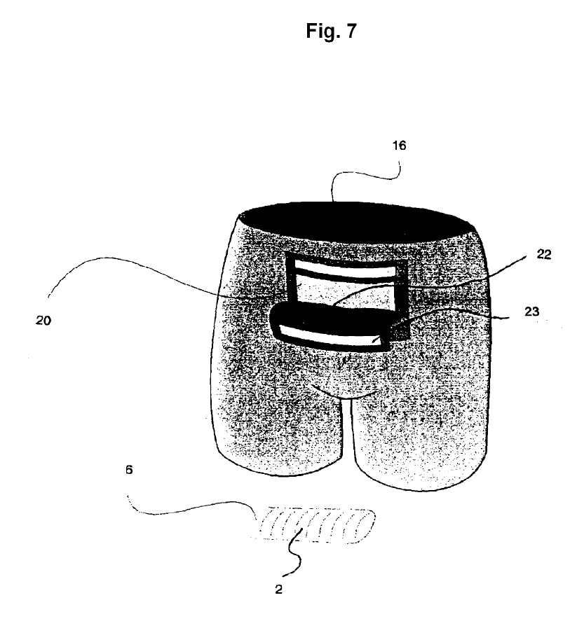

FIG. 7 shows an isometric view of how another embodiment of the therapeutic cooling or heating garment may be implemented. This embodiment is similar to the one shown in FIG. 6, with the difference being that the location of the pocket or sleeve 20 in this embodiment is near the lower abdominal region of the Lycra® underpants 16. FIG. 7 shows the pack 6 and the Lycra® underpants 16 in this embodiment as being separated before the therapeutic cooling or heating garment is assembled. The pocket or sleeve 20 may also include a closeable opening 22, which may be closed and re-opened using and adhesive closure tab or Velcro® tabs or dots 23.

Now that several preferred embodiments of the present invention have been described it will be apparent to those skilled in the art that the therapeutic cooling or heating pad or garment has the following advantages:

1. It may help relieve pain to various regions of the body after a person has given natural birth, had a caesarean section, or had surgery to the lower abdominal or pelvic region.

2. Lycra® underpants or undergarments or sanitary napkin made of relatively thin fabric are more effective in that the rate of heat transfer to the cooling agent (or from the heating agent) across the undergarment or the sanitary napkin is high.

3. The fact that the pack containing the cooling or heating agent is replaceable means that the undergarment or the sanitary napkin needs not be replaced should the need to switch to a different cooling or heating agent, or from a cooling to heating application, arise.

4. The cost of repeatedly using the therapeutic cooling or heating pad or garment may be reduced because the cooling or heating agent is reusable.

5. Since the pack containing the cooling or heating agent is removable from the undergarment or sanitary napkin, the undergarment or sanitary napkin can be laundered.

Those skilled in the art will appreciate that the invention described herein is susceptible to variations and modifications other than those specifically described. The undergarment is not restricted to Lycra® underpants but other undergarments, for example those made of other materials such as cotton, may also be used. The location of the pocket or sleeve may vary and is not restricted to the crotch, pelvic or lower abdominal areas. The undergarment or the sanitary pad may include more than more than one pocket or sleeve. For example, the embodiments illustrated in FIGS. 6 and 7 may be combined such that the undergarment may include a first pocket near the crotch area and a second pocket near the lower abdominal area. The cooling or heating agent may be integrated within the sanitary napkin or the undergarment by for example microencapsulation of the agent which is then dispersed through the sanitary napkin or undergarment.

All such variations and modifications are to be considered within the ambit of the present invention the nature of which is to be determined from the foregoing description.

1-27. (canceled)

28. A therapeutic cooling or heating pad comprising:
a sanitary napkin; and
one or more pockets attached to the sanitary napkin, the one or more pockets being configured to removably receive one or more packs each enclosing a cooling or heating agent, and being:
(i) in the form of a sleeve formed in the sanitary napkin and having a fastenable opening at one end; or
(ii) defined by a collapsible opening formed on an outward face of the sanitary napkin.

29. A therapeutic cooling or heating pad as claimed in claim 28, wherein the packs are disposable and/or replaceable.

30. A therapeutic cooling or heating pad as claimed in claim 28, wherein the cooling or heating agent is reusable.

31. A therapeutic cooling or heating pad as claimed in claim 28, wherein the cooling agent includes a cooling gel, ice, a phase change material or salt solution.

32. A therapeutic cooling or heating pad as claimed in claim 28, wherein the heating agent includes a phase change material or salt solution.
